(19) United States
(12) Patent Application Publication
(10) Pub. No.: US 2010/0137949 A1
(43) Pub. Date: Jun. 3, 2010

(54) BIFURCATED ELECTRICAL LEAD AND METHOD OF USE

(75) Inventors: Todor N. Mazgalev, Pepper Pike, OH (US); Youhua Zhang, Beachwood, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/471,686

(22) Filed: May 26, 2009

Related U.S. Application Data
(60) Provisional application No. 61/056,083, filed on May 27, 2008.

(51) Int. Cl. A61N 1/36; A61N 1/05

(52) U.S. Cl. 607/72; 607/116

(57) ABSTRACT

A bifurcated electrical lead includes an elongated lead body having a proximal end portion, a bifurcated distal end portion, and a main body portion extending between the proximal end portion and the bifurcated distal end portion. The bifurcated distal end portion includes oppositely disposed first and second arm members. The first and second arm members respectively include first and second electrodes operably coupled to first and second anchoring members. The first electrode is substantially parallel to the second electrode.

### RELATED APPLICATION

This application claims priority from U.S. Provisional Application No. 61/056,083, filed May 27, 2008, the subject matter of which is incorporated herein by reference.

### TECHNICAL FIELD

The present invention relates generally to an apparatus and method for neuromodulation, and more particularly to a bifurcated electrical lead and related method for modulating the electrical activity of a target tissue.

### BACKGROUND OF THE INVENTION

The automatic nervous system (ANS) regulates “involuntary” organs and maintains normal internal function and works with the somatic nervous system. The ANS includes the sympathetic nervous system (SNS) and the parasympathetic nervous system (PNS). The SNS is affiliated with stress and the “fight-or-flight response” to emergencies, and the PNS is affiliated with relaxation and the “rest-and-digest response.” Autonomic balance reflects the relationship between parasympathetic and sympathetic activity. Changes in autonomic balance are reflected in changes in heart rate, heart rhythm, contractility, remodeling, inflammation and blood pressure. Changes in autonomic balance can also be seen in other physiological changes, such as changes in abdominal pain, appetite, stamina, emotions, personality, muscle tone, sleep, and allergies, for example.

It is desirable to use a measurement of autonomic balance in order to appropriately control or titrate various neural stimulation therapies. Neural stimulators have been proposed to treat a variety of disorders, such as epilepsy, obesity, breathing disorders, hypertension, post-myocardial infarction (MI) remodeling and heart failure. Direct electrical stimulation has been applied to the carotid sinus and vagus nerve. Electrical stimulation of the carotid sinus nerve can result in reduction of experimental hypertension, and that direct electrical stimulation to the pressoreceptive regions of the carotid sinus itself brings about reflex reduction in experimental hypertension. Electrical systems have been proposed to treat hypertension in patients who do not otherwise respond to therapy involving lifestyle changes and hypertension drugs, and possibly to reduce drug dependency for other patients.

The stimulation of sympathetic afferents triggers sympathetic activation, parasympathetic inhibition, vasoconstriction, and tachycardia. In contrast, parasympathetic activation results in bradycardia, vasodilation and inhibition of vasopressin release. Direct stimulation of the vagal parasympathetic fibers has been shown to reduce heart rate. In addition, chronic stimulation of the vagus nerve may be of protective myocardial benefit following cardiac ischemic insult. Reduced autonomic balance (increase in sympathetic and decrease in parasympathetic cardiac tone) during heart failure has been shown to be associated with left ventricular dysfunction and increased mortality. Additionally, increasing parasympathetic tone and reducing sympathetic tone may protect the myocardium from further remodeling and predisposition to fatal arrhythmias following MI.

### SUMMARY OF THE INVENTION

According to one aspect of the present invention, a bifurcated electrical lead comprises an elongated lead body having a proximal end portion, a bifurcated distal end portion, and a main body portion extending between the proximal end portion and the bifurcated distal end portion. The bifurcated distal end portion includes oppositely disposed first and second arm members. The first and second arm members respectively include first and second electrodes operably coupled to first and second anchoring members. The first electrode is substantially parallel to the second electrode.

According to another aspect of the present invention, a method is provided for delivering electric current to a predetermined site comprising a target tissue. One step of the method includes providing a bifurcated electrical lead comprising an elongated lead body having a proximal end portion, a bifurcated distal end portion, and a main body portion extending between the proximal end portion and the bifurcated distal end portion. The bifurcated distal end portion includes oppositely disposed first and second arm members. The first and second arm members respectively include first and second electrodes operably coupled to first and second anchoring members. The first electrode is substantially parallel to the second electrode. The bifurcated distal end portion of the electrical lead body is delivered to the predetermined site, and the first and second arm members are then positioned so that the first and second electrodes are in electrical contact with the target tissue. Next, electric current is delivered to the first and second electrodes to modulate the electrical activity of the target tissue.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view of a bifurcated electrical lead constructed in accordance with the present invention;

FIG. 2 is a cross-sectional view of a human heart;

FIG. 3 is a perspective view showing the right side of the heart in FIG. 2;

FIG. 4 is a perspective view showing the posterior view of the heart in FIG. 2;

FIG. 5 is a process flow chart illustrating a method for delivering electric current to a predetermined site comprising a target tissue according to the present invention;

FIG. 6 is a perspective view showing a tissue penetrating portion of the bifurcated electrical lead in FIG. 1 being threaded through a cardiac fat pad;

FIG. 7A is a perspective view showing electrodes of the bifurcated electrical lead in FIG. 1 being embedded into the cardiac fat pad;

FIG. 7B is an exploded view of the cardiac fat pad in FIG. 7A showing the electrodes embedded in the cardiac fat pad; and

FIG. 8 is a perspective view showing a portion of the bifurcated electrical lead in FIG. 1 securely implanted within the cardiac fat pad.

### DETAILED DESCRIPTION

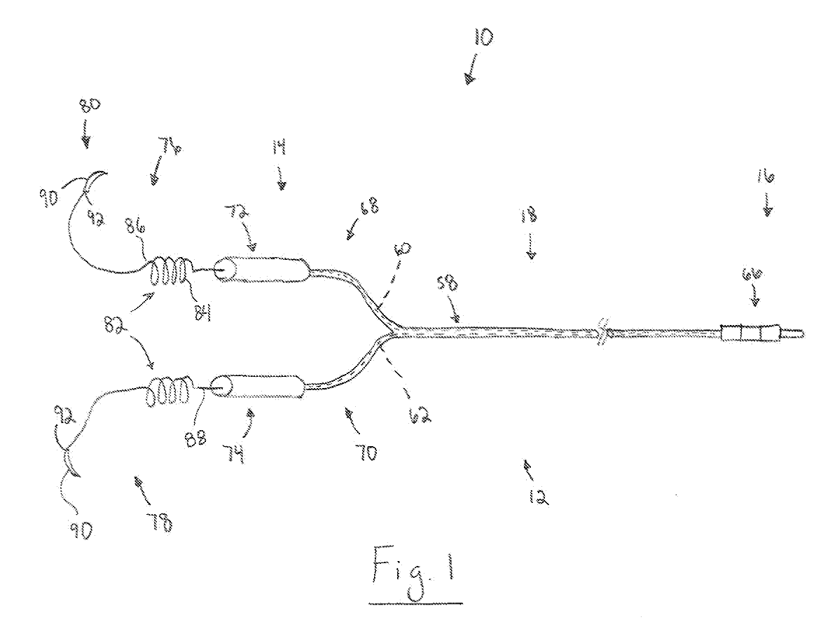

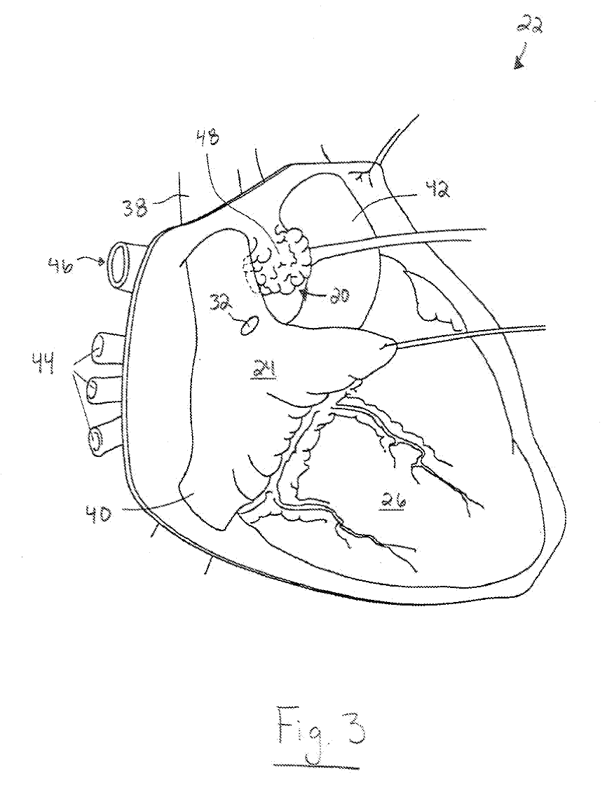

The present invention relates generally to an apparatus and method for neuromodulation, and more particularly to a bifurcated electrical lead and related method for modulating the electrical activity of a target tissue. As representative of the present invention, FIG. 1 illustrates a bifurcated electrical lead 10 comprising an elongated lead body 12 having a bifurcated distal end portion 14, a proximal end portion 16, and a main body portion 18 extending between the bifurcated distal end portion and the proximal end portion. Although the present invention is described in terms of using the bifurcated electrical lead 10 to deliver electric current to a cardiac fat pad 20 (FIG. 3), it will be appreciated that the present invention can be contacted with other biological tissue structures, such as those provided below.

Unless otherwise defined, all technical terms used herein have the same meaning as commonly understood by one of ordinary skill in the art to which the present invention pertains.

In the context of the present invention, the term “heart condition” can refer to a wide range of abnormalities and/or diseases of the heart, coronary vasculature, or blood vessels surrounding the heart, including underlying conditions, such as ischemia, atherosclerosis or coronary artery disease, embolism, congenital heart defects, anemia, lung disease, and abnormal stimulation (e.g., sympathomimetic abuse), hypertension (e.g., systemic hypertension, primary and secondary hypertension, pulmonary hypertension), chronic obstructive pulmonary disease, restrictive lung disease, pulmonary embolism, morbid obesity, valvular disease (e.g., mitral valve disease, aortic valve disease, tricuspid valve disease, and pulmonary valve disease), heart muscle disease (e.g., ischemic cardiomyopathy, dilated cardiomyopathy, hypertensive cardiomyopathy, hypertrophic cardiomyopathy, restrictive cardiomyopathy, and specific heart muscle disease resulting from cardiac infection), neuromuscular disease, storage disorders, infiltration disorders, immunologic disorders, pericardial disease, rheumatoid heart disease, neoplastic heart disease (e.g., primary cardiac tumors), coronary vasospasm (e.g., drug-induced vasospasm), cardiac trauma, genetic or hereditary predisposition that may manifest as angina (e.g., stable angina, unstable angina, mixed angina, and Prinzmetal's variant angina), myocardial infarction, chronic ischemic heart disease, and sudden cardiac death.

As used herein, the terms “modulate” or “modulating” can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, thermal, ultrasonic, optical or chemical, or a combination of two or more of these. The terms “modulate” or “modulating” can also be used to refer to a masking, altering, overriding, or restoring of neuronal activity.

As used herein, the term “target tissue” can refer to any portion of a human (or other mammalian) body that has been identified to benefit from receiving electric current. Non-limiting examples of target tissue can include biological tissue comprising at least one autonomic ganglion, such as a cardiac fat pad, a portion of the vagus nerve (e.g., the cervical vagus nerve), and any portion of the myocardium.

A brief discussion of the cardiac anatomy and physiology is provided to assist the reader with understanding the present invention. The automatic nervous system (ANS) regulates “involuntary” organs, while the contraction of voluntary (skeletal) muscles is controlled by somatic motor nerves. Examples of involuntary organs include respiratory and digestive organs, as well as blood vessels and the heart. Often, the ANS functions in an involuntary, reflexive manner to regulate glands, muscles in the skin, the eyes, stomach, intestines, bladder, cardiac muscles, and muscles surrounding blood vessels, for example.

The ANS includes the sympathetic nervous system (SNS) and the parasympathetic nervous system (PNS). The SNS is affiliated with stress and the “fight-or-flight response” to emergencies. Among other effects, the “fight-or-flight response” increases blood pressure and heart rate, thereby increasing skeletal muscle blood flow and decreasing digestion to provide energy for “fighting or fleeing.” The PNS is affiliated with relaxation and the “rest-and-digest response” which, among other effects, decreases blood pressure and heart rate, and increases digestion to conserve energy. The ANS maintains normal internal function and works with the somatic nervous system.

Electrically stimulating the SNS and PNS can have a number of physiological effects. For example, stimulating the SNS dilates the pupils, reduces saliva and mucus production, relaxes the bronchial muscle, reduces peristalsis of the stomach, increases the conversion of glycogen to glucose by the liver, decreases urine secretion by the kidneys, and closes the sphincter of the bladder. Stimulating the PNS constricts the pupils, increases saliva and mucus production, contracts the bronchial muscle, increases secretions and motility in the stomach and large intestine, increases digestion in the small intestine, increases urine secretion, and relaxes the sphincter of the bladder. The functions associated with the SNS and PNS can be integrated with each other. Thus, indiscriminate stimulation of the SNS and/or PNS to achieve a desired response, such as vasodilation, in one physiological system may also result in an undesired response in another physiological system.

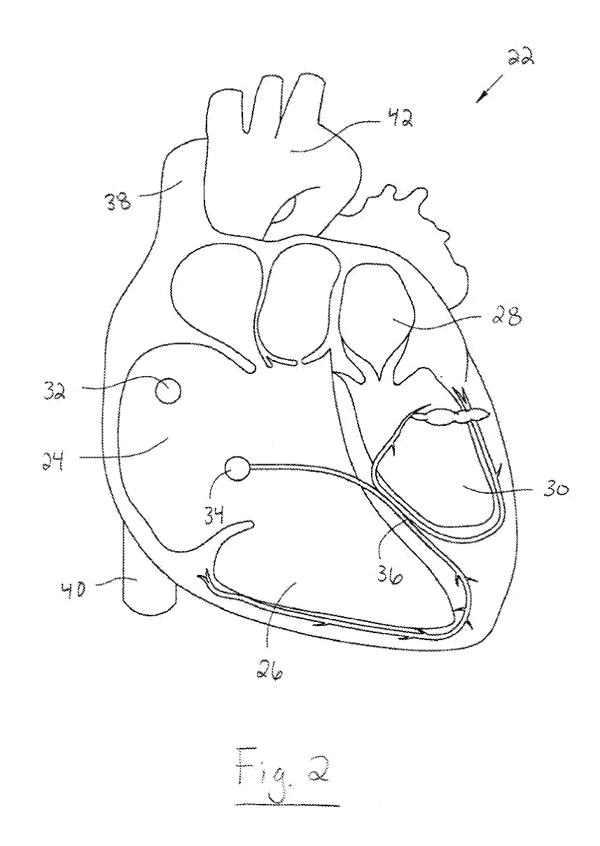

FIGS. 2-4 illustrate a human heart 22. The heart 22 includes a right atrium 24, a right ventricle 26, a left atrium (LA) 28, and a left ventricle 30. The heart 22 also includes a sinoatrial (SA) node 32 and an atrioventricular (AV) node 34. The SA node 32 comprises a cluster of cells in the right atrium 24 that generates electrical impulses. The AV node 34 comprises a cluster of cells situated in the center of the heart 22 between the atria 24 and 28 and the ventricles 26 and 30.

FIG. 2 illustrates the cardiac conduction system that controls heart rate. This system generates and conducts electrical impulses throughout the myocardium to stimulate cardiac constriction. The cardiac conduction system includes the SA node 32 and the AV node 34.

The ANS controls firing of the SA node 32 to trigger the start of the cardiac cycle. The electrical impulses generated by the SA node 32 are propagated between myocardial cells until the impulses reach the AV node 34. The AV node 34 functions as an electrical relay station between the atria 24 and 28 and the ventricles 26 and 30, such that electrical signals from the atria must pass through the AV node to reach the ventricles. The AV node 34 slows the electrical current before the signal is permitted to pass through the ventricles 26 and 30, thereby allowing the atria 24 and 28 to fully contract before the ventricles are stimulated. After passing the AV node 34, electrical impulses travel to the ventricles 26 and 30 along Purkinje fibers 36 embedded in the inner ventricular walls of the heart 22.

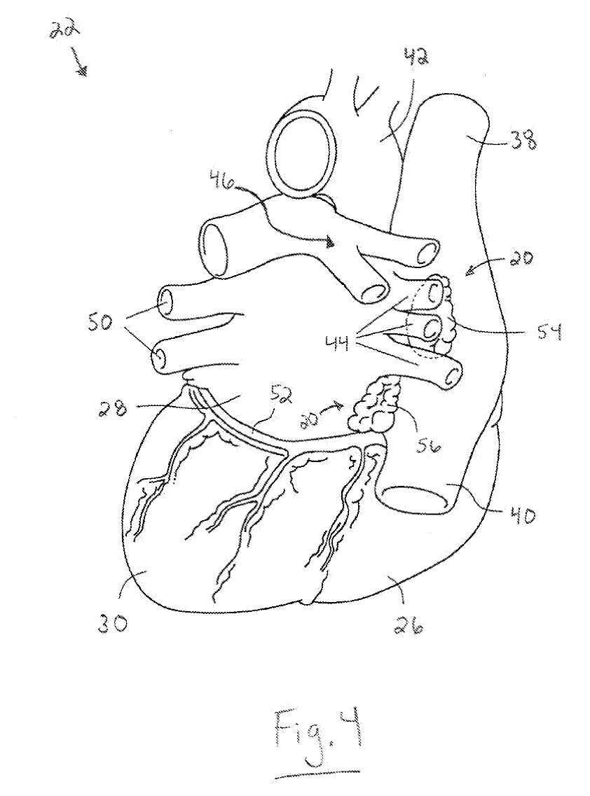

FIGS. 3-4 show the cardiac fat pads 20 of the heart 22. FIG. 3 shows the right atrium 24, right ventricle 26, the SA node 32, the superior vena cava (SVC) 38, the inferior vena cava (IVC) 40, the aorta (AO) 42, the right pulmonary veins 44, and the right pulmonary artery 46. FIG. 3 also shows a cardiac fat pad 20, referred to herein as the SVC-AO fat pad 48, located between the superior vena cava 38 and the aorta 42. FIG. 4 shows the LA 28, the left ventricle 30, the right ventricle 26, the SVC 38, the IVC 40, the AO 42, the right pulmonary veins 44, the left pulmonary veins 50, the right pulmonary artery 46, and the coronary sinus 52. FIG. 4 also shows a cardiac fat pad 20, referred to herein as the SA node (SN) fat pad 54, located proximate to a junction between the right atrium 24 and the right pulmonary veins 44. Additionally, FIG. 4 shows a cardiac fat pad 20, referred to herein as the IVC-LA fat pad 56, located proximate to or at the junction of the IVC 40 and the LA 28.

The SVC-AO fat pad 48 functions as a “head station” of vagal fibers (not shown) projecting to the right and left atria 24 and 28, the IVC-LA fat pad 56, and the SN fat pad 54. The portion of the ANS that regulates heart rhythm includes a number of ganglionated fat pads, i.e., the SVC-AO fat pad 48, the IVC-LA fat pad 56, and the SN fat pad 54. Parasympathetic ganglia in these cardiac fat pads 48, 56, and 54 exert important effects on chronotropy, dromotropy, and inotropy. For example, cardiac rate, AV conduction, and contractility are mediated through ganglia located in these cardiac fat pads 48, 56 and 54.

Disruption of neural activity in the cardiac fat pads 20 can cause significant heterogeneity of repolarization, and tends to result in atrial arrhythmias. An intrinsic cardiac neuronal network is important to both intracardiac and extracardiac integration of autonomic cardiac function. Unfortunately, this cardiac neuronal network can be damaged, thus adversely affecting the autonomic balance. For example, myocardial ischemia can compromise the function of neurons embedded with the cardiac fat pads 20, diabetic neuropathy can affect intrinsic cardiac innervation, and surgery may sever or otherwise damage a portion of the cardiac neural network.

Referring again to FIG. 1, one aspect of the present invention includes a bifurcated electrical lead 10 for modulating the electrical activity of a target tissue. The bifurcated electrical lead 10 comprises an elongated lead body 12 having a bifurcated distal end portion 14, a proximal end portion 16, and a main body portion 18 extending between the proximal end portion and the bifurcated distal end portion. The elongated lead body 12 can comprise any pair of suitable flexible electrical conductors 58, such as coaxial wires that are partially or entirely enveloped in one or more insulating materials. For example, the elongated lead body 12 can comprise a coaxial pair of helically-wound first and second electrical wires 60 and 62 made of multifilament or twisted stainless steel and respectively encased in first and second electrically insulative layers (not shown in detail). The elongated lead body 12 can have a rigid, semi-rigid, and/or flexible configuration and is capable of facilitating the flow of electrical current therethrough.

The first and second coaxial wires 60 and 62 can comprise twisted or helically-wound strands of medical grade stainless steel wire. Alternatively, the first and second coaxial wires 60 and 62 may be formed of single strands of stainless steel, or of one or more strands of electrically conductive polymeric material. The first insulative layer can be formed of fluorinated ethylene propylene (FEP), polytetrafluorethylene (PTFE), or any other suitable medical grade, biocompatible dielectric insulating coating, such co-polymer polytetrafluorethylene, polyethylene, silastic, neoprene, polypropylene, or polyurethane.

The second insulative layer can be comprised of at least one medical grade stainless steel strand or filament wound in helical fashion over the first insulative layer. Helical winding of the first and second insulative layers imparts a high degree of flexibility to the bifurcated electrical lead. The second insulative layer can be formed of FEP, polyethylene, or any other suitable biocompatible material, such as medical grade, biocompatible PTFE, polyethylene, silastic, neoprene, polypropylene, or polyurethane.

As shown in FIG. 1, both the main body portion 18 and the proximal end portion 16 of the elongated lead body 12 have a wire-like configuration.

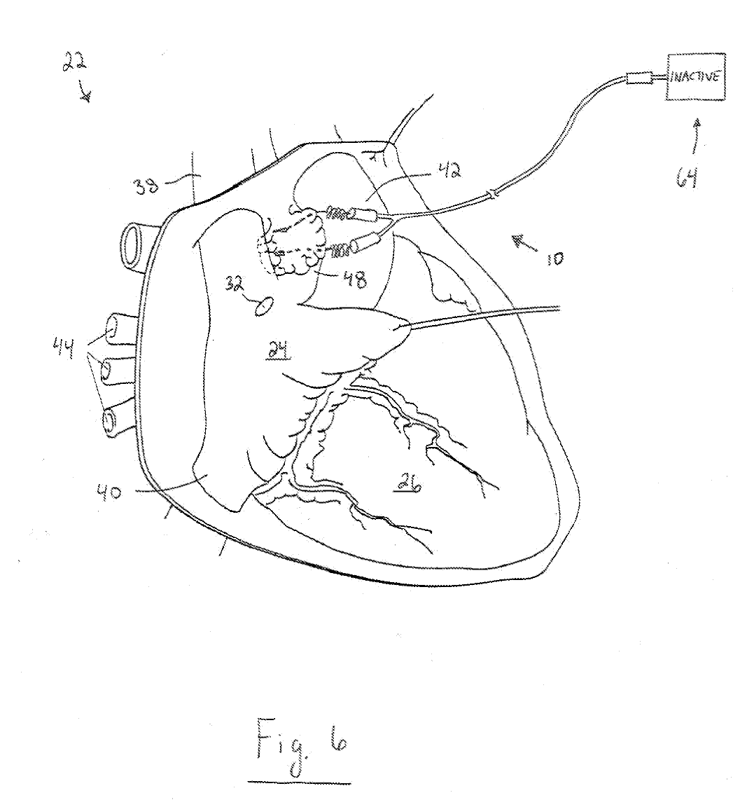

The proximal end portion 16 is adapted for connection to an energy delivery source 64 (FIG. 6) capable of delivering electric current to the bifurcated electrical lead 10 (FIG. 1). For example, the proximal end portion 16 can include an electrical connector 66, such as a bipolar IS-1 type lead connector configured for reception by the energy delivery source (e.g., an implantable pulse generator).

As shown in FIG. 1, the bifurcated distal end portion 14 includes oppositely disposed first and second arm members 68 and 70. The first and second arm members 68 and 70 respectively include first and second electrodes 72 and 74 integrally formed with first and second anchoring members 76 and 78. Each of the first and second arm members 68 and 70 also include a mono-polar conductor or wire 60 and 62 that electrically connects the first and second electrodes 72 and 74 with the energy delivery source 64.

The first electrode 72 is located substantially parallel to the second electrode 74. As explained in more detail below, the substantially parallel configuration of the first and second electrodes 72 and 74 can facilitate placement of the electrodes entirely within a target tissue. Unlike electrical leads of the prior art, which typically include two or more electrodes arranged in series, the substantially parallel arrangement of the first and second electrodes 72 and 74 allows the entirety of both electrodes to be placed in electrical contact with the target site without protruding from a portion of the target site.

As shown in FIG. 1, the first and second electrodes 72 and 74 have a cylindrical shape; however, it will be appreciated that the electrodes can have any shape and size including, for example, a triangular shape, a rectangular shape, or an ovoid shape. Although the bifurcated electrical lead 10 is shown with only first and second electrodes 72 and 74, it will be appreciated that any desired number of electrodes can be formed as part of the first and second arm members 68 and 70, so long as the electrodes are located substantially parallel from one another. The first and second electrodes 72 and 74 can be made of any material capable of conducting an electric current, such as titanium, platinum, platinum-iridium, and the like.

To facilitate focal delivery of electric current to a target tissue, the size and shape of the first and second electrodes 72 and 74 may be varied as needed. Additionally or optionally, the entire surface area of the first and second electrodes 72 and 74 may be conductive or, alternatively, only a portion of the surface area of the electrodes may be conductive. By modifying the size, shape, and conductivity of the surface of the first and second electrodes 72 and 74, the surface area(s) of the electrodes that contact a target tissue may be selectively modified to facilitate focal delivery of electric current. For example, electric current can be delivered to the first and second electrodes 72 and 74 such that the electric current is conducted only through selective portions of the electrodes. Delivery of electric current can then be selectively controlled or “titrated” to achieve a desired physiological effect.

The first and second anchoring members 76 and 78 are operably coupled to the first and second electrodes 72 and 74 (respectively). As described in more detail below, the first and second anchoring members 76 and 78 are for embedding into a tissue substrate, such as tissue surrounding or adjacent a target tissue. Each of the first and second anchoring members 76 and 78 comprises a tissue penetrating portion 80 and a tissue embedding portion 82. The tissue embedding portion 82 of each of the first and second anchoring members 76 and 78 includes first and second ends 84 and 86 and is adapted for placement in a substrate tissue. The first end 84 of each tissue embedding portion 82 is operably connected to the first and second electrodes 72 and 74. For example, the first end 84 of the tissue embedding portion 82 can be integrally formed within a portion of the first and second electrodes 72 and 74 or, alternatively, securely connected to an outer surface of the first and second electrodes.

The tissue embedding portion 82 of each of the first and second anchoring members 76 and 78 comprises a monofilament strand 88 formed of a biocompatible, medical grade polymer, such as polypropylene. The tissue embedding portion 82 has a spiral or pig tail-shaped configuration to facilitate fixation of the bifurcated electrical lead 10 in a target tissue, and prevent dislodgments that might otherwise occur were a smooth tipped electrical lead employed. It should be appreciated that the tissue embedding portion 82 can have any configuration that facilitates secure implantation of the first and second anchoring members 76 and 78 in a substrate tissue.

The tissue penetrating portion 80 of the first and second anchoring members 76 and 78 includes a curved needle 90 for piercing a target tissue and/or a substrate tissue. As shown in FIG. 1, the curved needle 90 includes a proximal end 92 connected to the monofilament strand 88 of the tissue embedding portion 82. It should be appreciated that the tissue penetrating portion 80 can have any desired shape or size besides (or in addition to) the curved needle 90 shown in FIG. 1. For example, the tissue penetrating portion 80 can have a barb or hook-shaped configuration. The tissue penetrating portion 80 can be made of any biocompatible, medical grade material, such as stainless steel.

It will be appreciated that the configuration of the first and second anchoring members 76 and 78 may be varied as needed. For example, the first anchoring member 76 may only include a tissue penetrating portion 80, while the second anchoring member 78 may include both a tissue embedding portion 82 and a tissue penetrating portion. Alternatively, each of the first and second anchoring members 76 and 78 may only include a tissue penetrating portion 80, or, each of the first and second anchoring members may only include a tissue embedding portion 82.

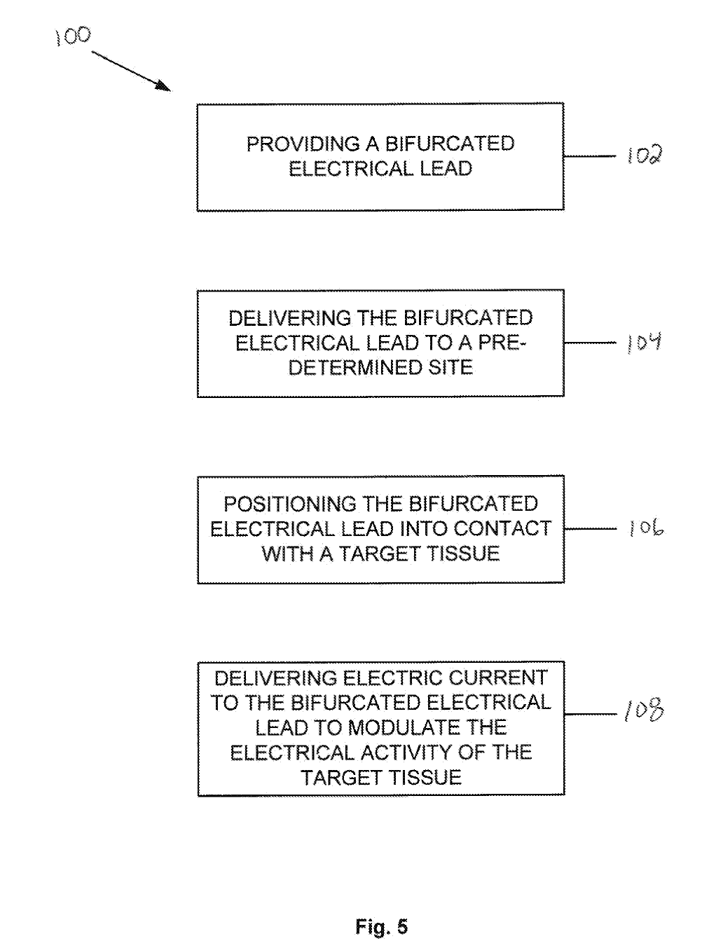

FIG. 5 is process flow diagram illustrating another aspect of the present invention. In FIG. 5, a method 100 is provided for delivering electric current to a predetermined site comprising a target tissue. The predetermined site comprises a portion of a mammalian subject, such as a human subject. Although the term “subject” as used herein typically refers to a human subject, it will be appreciated that the term can also include any warm-blooded organism including, but not limited to, pigs, rats, mice, dogs, goats, sheep, horses, monkeys, apes, rabbits, cattle, etc.

Generally, the method 100 of the present invention includes a neuromodulatory approach to treating one or a combination of cardiac conditions, such as those provided above. The present invention takes advantage of the substantially parallel configuration of the first and second electrodes 72 and 74 to accurately and selectively deliver electric current to a target tissue. By accurately and selectively delivering electric current to the first and second electrodes 72 and 74, the electrical activity of the target tissue can be modulated to affect the SNS, the PNS, or both.

At 102, one step of the method 100 includes providing a bifurcated electrical lead 10 as shown in FIG. 1 and as described above. The bifurcated electrical lead 10 is delivered to a predetermined site comprising a target tissue at 104. As noted above, the target tissue can include any portion of a human (or other mammalian) body that has been identified to benefit from receiving electric current. The target tissue can include, for example, any portion of the SNS, the PNS, or both, such as biological tissue comprising at least one autonomic ganglion (e.g., a cardiac fat pad 20 or a portion of the vagus nerve) or muscle tissue (e.g., a portion of the myocardium).

In an example of the method 100, the predetermined site can include any one or combination of cardiac fat pads 20. As noted above, the cardiac fat pads 20 contain ganglia that innervate the heart 22 and can include, for example: the SN fat pad 54, which supplies nerve fibers to the superior right atrium 24 and the SA node 32; the IVC-LA fat pad 56, which supplies nerve fibers to the AV node 34 region and both atria; and the SVC-AO fat pad 48, located between the SVC 38 and the AO 42. The SVC-AO fat pad 48 provides efferent fibers to both the SN and IVC-LA fat pads 54 and 56, as well as additional fibers to both atria 24 and 28. The SN, SVC-AO, and IVC-LA fat pads 54, 48, and 56 are of particular interest because they are accessible and distinctly identifiable. For example, the SN fat pad 54 and the IVC-LA fat pad 56 may serve as the predetermined site since efferent fibers from the SVC-AO fat pad 48 are provided to them as well.

At 104, the bifurcated electrical lead 10 is delivered to the predetermined site using any appropriate surgical approach, such as an epicardial or endocardial approach. For example, the bifurcated electrical lead 10 can be delivered to the predetermined site during an open-chest surgical procedure or via a percutaneous transluminal procedure. It will be appreciated that delivery of the bifurcated electrical lead 10 may also be by any transthoracic, minimally invasive technique known in the art.

At 106, the bifurcated electrical lead 10 is positioned at the predetermined site such that the first and second electrodes 72 and 74 are in electrical contact with the target tissue. By “electrical contact” it is meant that when electric current is delivered to the first and second electrodes 72 and 74, deplorization of at least one nerve comprising the target tissue is elicited. The first and second electrodes 72 and 74 can be placed directly on a surface of the target tissue, within all or an entire portion of the target tissue, or in close proximity to the target tissue but without being in direct contact with the target tissue.

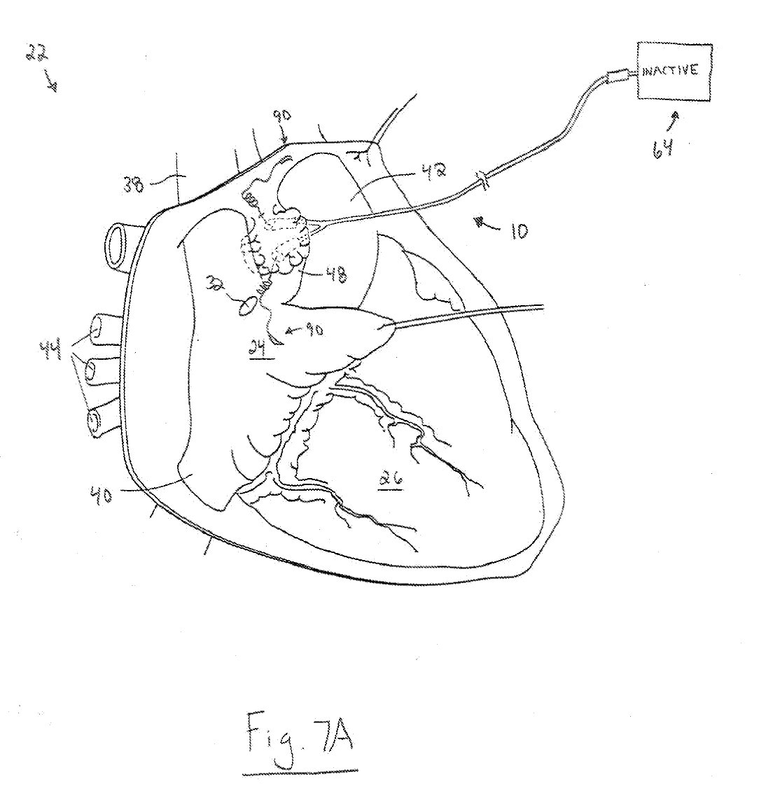

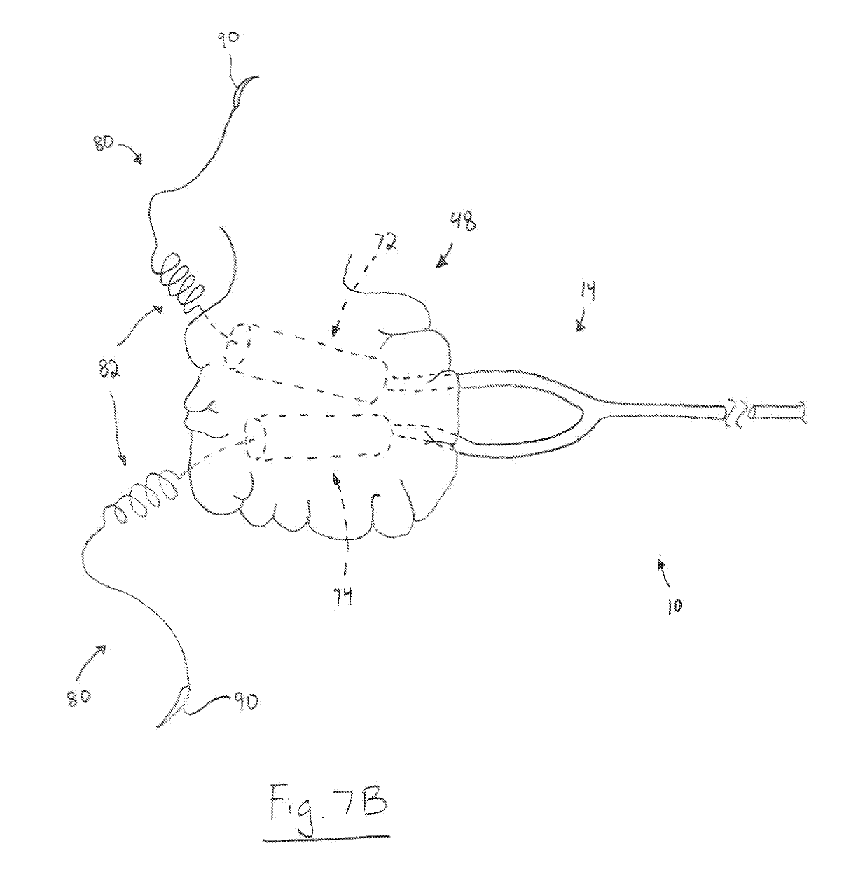

In one example of the method 100, the bifurcated distal end portion 14 of the bifurcated electrical lead 10 is positioned adjacent an SVC-AO fat pad 48 at 106. As shown in FIG. 6, the curved needle 90 of each of the first and second anchoring members 76 and 78 is first threaded through the SVC-AO fat pad 48. Next, the curved needles 90 are manipulated so that both the curved needles and the tissue embedding portion of the first and second anchoring members 76 and 78 are pulled through the SVC-AO fat pad 48 (FIG. 7A). As shown in FIG. 7B, the first and second anchoring members 76 and 78 are then pulled through the SVC-AO fat pad 48 until each of the first and second anchoring members protrude from the SVC-AO fat pad and the first and second electrodes 72 and 74 are entirely embedded within the SVC-AO fat pad tissue.

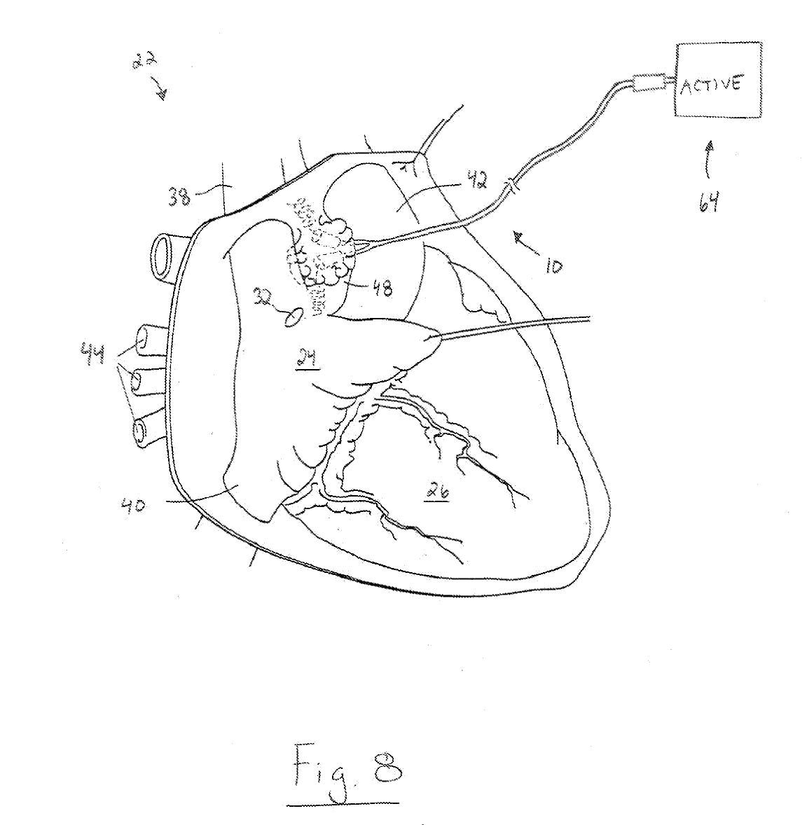

After positioning the first and second electrodes 72 and 74 so that the electrodes are in electrical contact with the SVC-AO fat pad 48, the curved needles 90 are threaded into a substrate tissue surrounding the SVC-AO fat pad (e.g., a portion of the myocardium). The curved needles 90 are then pulled through the myocardium so that the tissue embedding portion 82 of each of the first and second anchoring members 76 and 78 is embedded into a portion of the myocardium (FIG. 8). After the tissue embedding portion 82 of each of the first and second anchoring members 76 and 78 is secured within the myocardium, the curved needles 90 (and any portion of the tissue embedding portions) that extend from the myocardium are clipped off with scissors (not shown).

At 108, electric current is then delivered to the first and second electrodes 72 and 74 to modulate the electrical activity of the target tissue. Electric current is delivered to the first and second electrodes 72 and 74 using any one or combination of known energy delivery sources 64, such as an implantable pulse generator. Generally, the energy delivery source 64 can include any device capable of providing selective neuromodulation to pace the heart 22, improve contractility, and provide a stimulus to improve pumping efficiency and/or cardiac output.

Electric current can be delivered to the first and second electrodes 72 and 74 using a variety of internal, passive, or active energy delivery sources 64. The energy delivery source 64 may include, for example, radio frequency energy, X-ray energy, microwave energy, acoustic or ultrasound energy, such as focused ultrasound or high intensity focused ultrasound energy, light energy, electric field energy, thermal energy, magnetic field energy, combinations of the same, or any other energy delivery source used with implantable pulse generators known in the art. The energy delivery source 64 can be directly or indirectly (e.g., wirelessly) coupled to the bifurcated electrical lead 10.

Electric current can be delivered to the first and second electrodes 72 and 74 continuously, periodically, episodically, or a combination thereof. For example, electric current can be delivered in a unipolar, bipolar, and/or multipolar sequence or, alternatively, via a sequential wave, charge-balanced biphasic square wave, sine wave, or any combination thereof. Electric current can be delivered to the first and second electrodes 72 and 74 all at once or, alternatively, to only one of the electrodes using a controller (not shown) and/or known complex practice, such as current steering.

The particular voltage, current, and frequency delivered to the first and second electrodes 72 and 74 may be varied as needed. For example, electric current can be delivered to the first and second electrodes 72 and 74 at a constant voltage (e.g., at about 0.1 v to about 25 v), at a constant current (e.g., at about 25 microampes to about 50 milliamps), at a constant frequency (e.g., at about 5 Hz to about 10,000 Hz), and at a constant pulse-width (e.g., at about 50 μsec to about 10,000 μsec).

The bifurcated electrical lead 10 can be part of an open- or closed-loop system. In an open-loop system, for example, a physician may, at any time, manually or by the use of pumps, motorized elements, etc. adjust treatment parameters such as pulse amplitude, pulse width, pulse frequency, or duty cycle. Alternatively, in a closed-loop system, electrical parameters may be automatically adjusted in response to a sensed symptom or a related symptom indicative of the extent of the cardiac condition being treated. In a closed-loop feedback system, a sensor (not shown) that senses a condition (e.g., a metabolic parameter of interest, such as vagal activity) of the body can be utilized. More detailed descriptions of sensors that may be employed in a closed-loop system, as well as other examples of sensors and feedback control techniques that may be employed are disclosed in U.S. Pat. No. 5,716,377, which is hereby incorporated by reference in its entirety.

Delivery of electric current to the first and second electrodes 72 and 74 can stimulate or inhibit the SNS or, alternatively, stimulate or inhibit the PNS. For example, delivery of electric current can increase the amount of sympathetic nerve traffic to the myocardium to treat conditions in which an increase in heart rate or an increase in the inotropic state of the heart is desirable (e.g., bradycardia and acute cardiac failure). Alternatively, selective stimulation of epicardial autonomic ganglia can be used to selectively activate the PNS. For example, electric current can be delivered to a target tissue to modulate postganglionic parasympathetic nervous system activity and thereby decrease or increase left ventricular activity.

Stimulation of one or more cardiac fat pads 20 can directly affect cardiac tissue as fat pad ganglia form part of the parasympathetic efferent pathway. Where the first and second electrodes 72 and 74 of the bifurcated electrical lead 10 are positioned in the SVC-AO fat pad 48 (FIGS. 7A-B), for example, delivery of electric current increases vagal activity and thereby activates parasympathetic efferents. More particularly, delivery of electric current to the first and second electrodes 72 and 74 stimulates the parasympathetic efferents, thereby reducing contractility of the left ventricle 30 and providing a treatment for different cardiac conditions, such as heart failure and/or post-myocardial infarction remodeling.

It should be appreciated that the method 100 of the present invention can be used to modulate the electrical activity of cardiac fat pads 20 other than the SVC-AO fat pad 48. For example, the bifurcated electrical lead 10 can be positioned to deliver electric current to the SN fat pad 54 or the IVC-LA fat pad 56. Stimulation of the SN fat pad 54 can reduce the sinus rate, and stimulation of the IVC-LA fat pad 56 can increase AV conduction and thereby affect timing between contractions in the right atrium 24 and the right ventricle 26.

It should also be appreciated that electric current can additionally or optionally be applied to a portion of the myocardium, depending upon the clinical need(s) of the subject. For example, electric current may be applied directly or indirectly to a portion of the myocardium. Direct or indirect delivery of electric current to a portion of the myocardium can stimulate autonomic nerves innervating the myocardium without eliciting depolarization and contraction of the myocardium directly because the threshold for neural depolarization (especially myelinated vagal nerve fibers of the parasympathetic nervous system) is much lower than that of myocardial tissue. Differing frequencies of electrical stimulation can be used so as to depolarize pre- and post-ganglionic nerve fibers. For example, a stimulus response curve may be generated to determine the minimal threshold required to elicit myocardial contraction and still maintain neural depolarization at the stimulation site.

From the above description of the invention, those skilled in the art will perceive improvements, changes and modifications. For example, it should be appreciated that the first and second anchoring members 76 and 78 can be implanted entirely within the substrate tissue, partially within the substrate tissue (i.e., partly in the target tissue and partly in the substrate tissue), or entirely within the target tissue (FIG. 8). Such improvements, changes, and modifications are within the skill of the art and are intended to be covered by the appended claims.

Having described the invention, we claim:

1. A bifurcated electrical lead comprising:
an elongated lead body having a proximal end portion, a bifurcated distal end portion, and a main body portion extending between said proximal end portion and said bifurcated distal end portion;
said bifurcated distal end portion including oppositely disposed first and second arm members, said first and second arm members respectively including first and second electrodes operably coupled to first and second anchoring members, said first electrode being substantially parallel to said second electrode.

2. The apparatus of claim 1, wherein said first and second electrodes are for delivering electric current to a target tissue.

3. The apparatus of claim 1, wherein said first and second anchoring members are for embedding into a tissue substrate.

4. The apparatus of claim 1, wherein each of said first and second anchoring members further comprise a tissue penetrating portion and a tissue embedding portion.

5. The apparatus of claim 4, wherein said tissue embedding portion has a spiral configuration.

6. The apparatus of claim 1, wherein said proximal end portion of said electrical lead body includes an electrical connector adapted for connection to an implantable pulse generator.

7. The apparatus of claim 1, wherein said first and second electrodes are adapted for placement at a predetermined site comprising at least one autonomic ganglion.

8. The apparatus of claim 7, wherein the predetermined site is selected from the group consisting of a cardiac fat pad and a portion of the cervical vagus nerve.

9. The apparatus of claim 1, wherein said first and second electrodes are adapted for placement at a predetermined site comprising a portion of the myocardium.

10. The apparatus of claim 1, wherein said first and second branch members each comprise a mono-polar electrical wire.

11. A method for delivering electric current to a predetermined site comprising a target tissue, said method comprising the steps of:
providing a bifurcated electrical lead comprising an elongated lead body having a proximal end portion, a bifurcated distal end portion, and a main body portion extending between the proximal end portion and the bifurcated distal end portion, the bifurcated distal end portion including oppositely disposed first and second arm members, the first and second arm members respectively including first and second electrodes operably coupled to first and second anchoring members, the first electrode being substantially parallel to the second electrode;
delivering the bifurcated distal end portion of the electrical lead body to the predetermined site;
positioning the first and second arm members so that the first and second electrodes are in electrical contact with the target tissue; and
delivering electric current to the first and second electrodes to modulate the electrical activity of the target tissue.

12. The method of claim 11, wherein said step of delivering the bifurcated distal end portion of the electrical lead body to the predetermined site further includes securing the first and second electrodes at the predetermined site.

13. The method of claim 12, wherein said step of securing the first and second electrodes at the predetermined site further includes embedding the first and second anchoring members in a substrate tissue.

14. The method of claim 11, wherein the target tissue includes at least one autonomic ganglion.

15. The method of claim 14, wherein the target tissue is selected from the group consisting of a portion of the cervical vagus nerve and a cardiac fat pad.

16. The method of claim 11, wherein the target tissue includes a portion of the myocardium.

17. The method of claim 11, wherein the first and second electrodes are entirely contained within the target tissue.

18. The method of claim 11, wherein said step of delivering electric current to the predetermined site to modulate the electrical activity of the target tissue further comprises the steps of:
connecting the proximal end portion of the electrical lead body to an implantable pulse generator; and
causing the implantable pulse generator to deliver electric current to the first and second electrodes.
